(19) United States
(12) Patent Application Publication
(10) Pub. No.: US 2022/0330858 A1
(43) Pub. Date: Oct. 20, 2022

(54) BLOOD OPTIMIZATION SET WITH TRANSFER DEVICE

(71) Applicant: Kurin, Inc., San Diego, CA (US)

(72) Inventors: Thomas Thompson, Woodbury, MN (US); Kevin Nason, Phoenix, AZ (US); Bobby E. Rogers, Park City, UT (US); Jeffrey VELOZ, San Diego, CA (US)

(21) Appl. No.: 17/696,860

(22) Filed: Mar. 17, 2022

Related U.S. Application Data
(60) Provisional application No. 63/161,906, filed on Mar. 16, 2021.

(51) Int. Cl. A61B 5/153; A61B 5/15

(52) U.S. Cl. CPC A61B 5/153; A61B 5/15003; A61B 5/150343

(57) ABSTRACT

A bodily fluid collection system for collecting a bodily fluid from a patient, and a bodily fluid transfer system for transferring collected bodily fluid to a second collection device. A collection adapter has a connection port configured to securely connect with a hub of a syringe, and a collection interface opposite the connection port creates a fluid conduit to the barrel via the connection port and an inlet of the hub. A proximal adapter is connected to a proximal end of the fluid channel from the patient, and configured to removably interact with the collection interface of the collection adapter to connect the proximal end of the fluid channel to the barrel via the fluid conduit and inlet to the barrel to allow conveyance into the barrel of the at least some of the sampling portion of the sample of bodily fluid. The proximal adapter can be removed to allow access to any bodily fluid collected by the syringe.

### BACKGROUND

The present invention relates to bodily fluid culture contamination reduction systems, and more particularly a system and method for reducing bodily fluid culture contamination when using a syringe for a bodily fluid draw.

Certain bodily fluids can become contaminated. For instance, a condition known as bacteraemia is the presence of microorganisms in the blood. Sepsis, on the other hand, is bacteraemia in the presence of clinical symptoms and signs such as fever, tachycardia, tachypnea and hypotension. Bacteraemia and sepsis are associated with a high mortality and an increased incidence and duration of hospital stay and associated costs. Many bacteraemias, sepsis, fungaemias (fungal contamination of a bodily fluid) and other pathogens actually originate from a hospital or other healthcare settings, with catheters and venipunctures being a major source of contamination as potential carriers of these pathogens.

Where the bodily fluid is blood, blood cultures are the standard test used to detect microbial pathogens related to bacteraemia and sepsis in a patient's blood. The term blood culture refers to a single venipuncture, either from a peripheral site or central or arterial line, with the blood inoculated into one or more blood culture bottles or containers. One bottle is considered a blood culture where two or more are considered a set. Multiple sets may be obtained from multiple venipunctures and are associated with different sites on the patient. These methods allow for microbial identification and susceptibility testing to be performed, which is a critical component to managing sepsis, however the lack of rapid results and decreased sensitivity for fastidious pathogens has led to the development of improved systems and adjunctive molecular or proteomic testing.

Collection of bodily fluid samples for conducting fluid cultures is a critical component of modern patient care and can either positively affect the patient outcome by providing an accurate diagnosis, or, by causing an infection or indicating a false positive diagnosis, can adversely affect the outcome by prolonging unnecessary antimicrobial therapy, the length of hospital stays, and increasing costs.

One outcome of collection of blood cultures is contamination. Blood culture contamination can lead to a false positive culture result and/or significant increase in healthcare related costs. Sources of blood culture contamination include improper skin antisepsis, improper collection tube disinfection, and contamination of the initial blood draw which may then skew results.

Fluid culture collection kits generally consist of a “butterfly” set, infusion set, or other type of venipuncture device, and aerobic and anaerobic blood culture bottles. Various different bottles are also available depending on the test requirements. These bottles are specifically designed to optimize recovery of both aerobic and anaerobic organisms. In conventional kits, a bottle used is known generally as a “Vacutainer,” which is a blood collection tube formed of a sterile glass or plastic tube with a closure that is evacuated to create a vacuum inside the tube to facilitate the draw of a predetermined volume of liquid such as blood.

False positive blood cultures are typically a result of poor sampling techniques. They cause the use of antibiotics when not needed, increasing hospital costs and patient anxiety. Blood cultures are drawn from a needlestick into the skin, and then a Vacutainer is attached to capture a sample of blood. Contamination may occur from improper or incomplete disinfection of the skin area in and around the puncture site. It may also occur from the coring of the skin by the needle during insertion, with the cored skin cells and any associated contamination being pulled into the sample.

Blood flow through a hypodermic needle is laminar, and as such, a velocity gradient can be developed over the flow tube as a pressure drop is applied to the hypodermic needle. Either forceful aspiration of blood, or using a very small hypodermic needle, can cause lysis and a release of potassium from the red blood cells, thereby rendering the blood samples abnormal. In other instances, some patients have delicate veins that can collapse under a pressure drop or vacuum, particularly as applied by a syringe's plunger that is drawn too quickly for the patient's condition. Since such condition is impossible to know beforehand, such vein collapses are a risk and very difficult to control. In yet other instances, a syringe is used for a blood draw if a patient's blood pressure is extremely low, as in the case of infants or neonatal patients. The syringe can be connected with the venipuncture needle via tubing and/or other fluid-conveying components.

Various strategies have been implemented to decrease culture contamination rates, e.g. training staff with regard to aseptic collection technique, feedback with regard to contamination rates and implementation of blood culture collection kits. Although skin antisepsis can reduce the burden of contamination, 20% or more of skin organisms are located deep within the dermis and are unaffected by antisepsis or other cleaning, such as swabbing by an alcohol pad. Some conventional systems and techniques for reducing blood culture contamination include discarding the initial aliquot of blood taken from central venous catheters, venipunctures, and other vascular access systems. However, these systems require the user to mechanically manipulate an intravascular device, or require a complex series of steps that are difficult to ensure being followed.

As alluded to above, in cases where a syringe is used for a blood or other fluid draw from a patient, a transfer device may be needed. The transfer device facilitates transfer from the syringe into a collection container such as a Vacutainer® bottle or other container, but only after the transfer device is disconnected from a fluid collection set. However, once a needle set or its tubing is disconnected, if the connection with the contents of the syringe are not closed, there could be inadvertent, and possibly serious, fluid leakage that could negate the effectiveness of the entire draw.

### SUMMARY

This document discloses a transfer system for a fluid collection set. The transfer system includes a syringe for drawing bodily fluids from a patient via a sealed and secured connector or proximal adapter, and a collection adapter that accepts a collection device such as a bottle or tube for transferring the drawn bodily fluids from the syringe to the collection device once the proximal adapter is disconnected. The proximal adapter and the connection between the syringe and the collection adapter is configured to remain sealed after the proximal adapter is disconnected, and until a collection device is received by the collection adapter.

In some aspects, a bodily fluid collection system for collecting a bodily fluid from a patient is disclosed. The bodily fluid collection system includes a patient needle for venipuncture of the patient to access a sample of the bodily fluid from the patient, and a fluid channel having a distal end connected with the patient needle to convey at least a sampling portion of the sample of the bodily fluid accessed from the patient. The system further includes a syringe having a barrel to contain bodily fluid, a plunger that can be activated to produce a vacuum in the barrel to draw at least some of the sampling portion of the bodily fluid into the barrel, and a hub opposite the plunger to create an inlet into the barrel. The system further includes a collection adapter having a connection port configured to securely connect with the hub of the syringe, and a collection interface opposite the connection port that creates a fluid conduit to the barrel via the connection port and the inlet of the hub. The system further includes a proximal adapter connected to the proximal end of the fluid channel, the proximal adapter being configured to removably interact with the collection interface of the collection adapter to connect the proximal end of the fluid channel to the barrel via the fluid conduit and inlet to the barrel to allow conveyance into the barrel of the at least some of the sampling portion of the sample of bodily fluid.

In other aspects, A bodily fluid transfer system for transferring a sampling portion of a bodily fluid collected by a syringe from a fluid channel, the syringe having a barrel to contain bodily fluid, a plunger that can be activated to produce a vacuum in the barrel to draw at least some of the sampling portion of the bodily fluid into the barrel, and a hub opposite the plunger to create an inlet into the barrel.

The bodily fluid transfer system includes a collection adapter having a connection port configured to securely connect with the hub of the syringe, and a collection interface opposite the connection port that creates a fluid conduit with the barrel via the connection port and the inlet of the hub. The bodily fluid transfer system further includes a proximal adapter connected to a proximal end of the fluid channel, the proximal adapter being configured to removably interact with the collection interface of the collection adapter to connect the proximal end of the fluid channel to the barrel via the fluid conduit and inlet to the barrel to allow conveyance into the barrel of the at least some of the sampling portion of the sample of bodily fluid, and upon removal of the proximal adapter from the collection interface, allow transfer of at least some of the sampling portion of the sample of the bodily fluid in the barrel to a second collector connected with the collection interface of the collection adapter.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 is a block diagram of a bodily fluid collection system having a transfer device for transfer of fluids from a syringe to a collection bottle;

FIG. 2 illustrates a bodily fluid collection system with transfer system and device;

FIG. 3 illustrates a transfer system with a proximal adapter;

FIG. 4 is a perspective view of the proximal adapter of FIG. 3;

FIGS. 5(A) and 5(B) illustrates an implementation of a proximal adapter having a self-sealing septum;

FIG. 6 shows another implementation of a proximal adapter having one or more clips that clip around an outward-extending lip at the opening of a collection adapter;

FIG. 7 shows another implementation of a proximal adapter having a threaded ring that is adapted to be twisted onto and off a collection adapter;

FIGS. 8(A) and 8(B) show the proximal adapter of FIG. 7, both in its disconnected state and in its connected state, respectively;

FIG. 9 shows a transfer system having an external valve to open and close flow between a syringe and a collection adapter;

FIG. 10 shows a three-way stopcock with a needle-free connector added inline of, or between, a needle set and a collection device; and

FIG. 11 shows a three-way male connector and a needleless connector added inline of, or between, a needle set and a collection device.

Like reference symbols in the various drawings indicate like elements.

### DETAILED DESCRIPTION

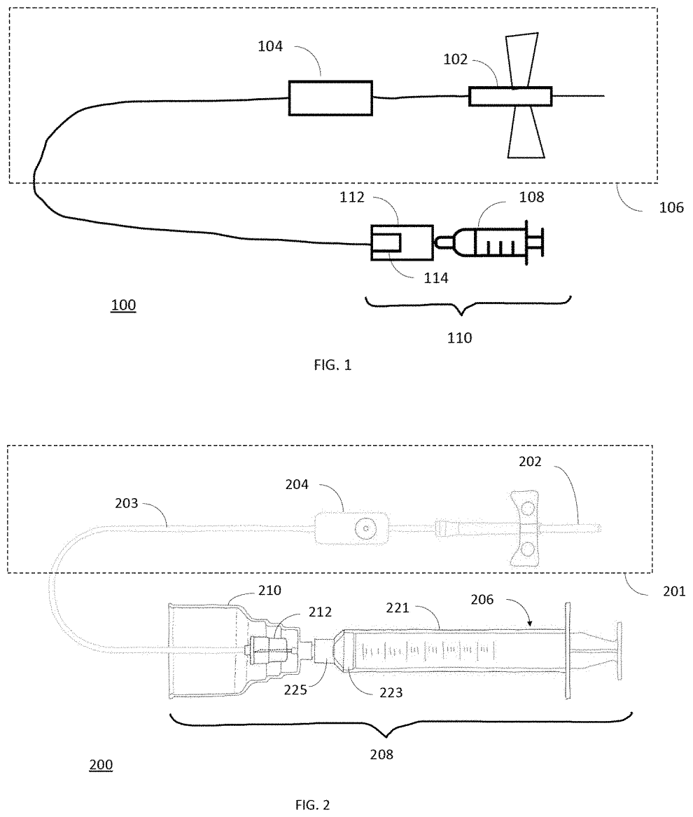

This document describes a bodily fluid collection system 100 as illustrated in FIG. 1, the bodily fluid collection system 100 having a fluid access device 102 such as a needle set for a venipuncture (such as described in U.S. Patent Publication No. 2019/0030293, the contents of which are incorporated by reference herein for all purposes), a fluid sample optimization device 104 such as a Kurin® lock (described in U.S. Patent Publications 2017/0020427, 2017/0020428, 2018/0177445, 2018/0271425, 2018/0289304, 2019/0159711, 2020/00281514 and 2020/00305780, the contents of each of which are incorporated by reference herein for all purposes). The fluid access device 102 and the fluid sample optimization device 104, along with associated tubing or fluid conduits connected to them, are sometimes referred to herein simply as a “set,” illustrated as set 106.

The bodily fluid collection system 100 further includes a fluid collection device such as a syringe 108 that provides the drawing or vacuum force through the fluid optimization device 104 and to the fluid access device 102. The syringe 108 can also be part of a transfer device or system 110 that can include a collection adapter 112 connected with the syringe 108 to allow two way fluid flow therebetween based on operation of the syringe 108, and proximal adapter 114 that securely connects the set 106 to the collection adapter 112 and transfer system 110, as will be explained further below.

In some implementations, for example, a fluid sample optimization device 104 for optimizing a fluid sample collected by a fluid collection device from a fluid source, where a first portion of the fluid sample potentially likely has contaminants, includes a housing defining an inlet port, an outlet port, a contaminant containment chamber connected with the inlet port, and a sampling channel having a proximal end connected with the inlet port and a distal end connected with the outlet port. The contaminant containment chamber includes a vent configured to allow air to be displaced therein by a first portion of blood entering the inlet port and into the contaminant containment chamber such that a second portion of blood flows into the sampling channel past the contaminant containment chamber and to the outlet port. In some particular implementations, the vent can include a plug to enclose the vent to inhibit air from entering the contaminant containment chamber. In yet other implementations, the vent forms the plug to close when contacted by a bodily fluid such as blood.

FIG. 2 illustrates a bodily fluid collection system 200 having a set 201 that includes a patient needle 202 for venipuncture of a patient and conveying a fluid sample from the patient to a fluid channel 203. The system 200 further includes a fluid sample optimization device 204 connected with the patient needle 202 for optimizing a fluid sample conveyed by the patient needle by sidelining and at least temporarily containing contaminants in a first portion or aliquot of the fluid sample and allowing subsequent, contaminant-free portions of the fluid sample to pass to a fluid collector such as a syringe 206.

The syringe 206 includes a barrel 221 to contain bodily fluid, a plunger 223 that can be activated to produce a vacuum in the barrel 221 to draw at least some of the sampling portion of the bodily fluid into the barrel 221, and a hub 225 opposite the plunger 223 to create an inlet into the barrel.

The fluid sample optimization device 204 can be connected to the patient needle by tubing, Luer connector, or the like, and the syringe 206 can be connected with the fluid sample optimization device 204 also by tubing or other fluid conveyance device.

Where the syringe 206 is used to draw fluid through the needle 202 and the fluid sample optimization device 204, it is common that the drawn fluid needs to be transferred to another collection device, such as a Vacutainer® bottle or the like. Accordingly, the bodily fluid collection system 200 includes a transfer system 208 that includes the syringe 206 and a collection adapter 210 attached to the opening of the syringe. The collection adapter 210 connects to the syringe by a connection port, such as a Luer connector, and includes a housing and an opening opposite the connection port that is sized and adapted for receiving a collection bottle or tube, such as an evacuated bottle. In some implementations, the collection adapter further includes a collection needle, initially connected with fluid sample optimization device 204 via proximal tubing or other fluid conduit and a proximal adapter 212, and through which at least some of the subsequent portions of fluid drawn by the syringe 206 are conveyed to the syringe 206. The proximal adapter 212 can be part of the set 201 as the proximal end of tubing connected with the fluid sample optimization device 204.

The proximal adapter 212 can be a self sealing membrane or pre slit membrane and may include a Luer connection, a snap arm, twist, friction fit, or other securement device for securely attaching the set 201 to the transfer system 208, via the collection adapter. The proximal adapter 212 seals to the transfer system 208 to allow fluid to flow into the transfer system 208 and to the syringe 206.

Importantly, the set 201 needs a way or mechanism to seal once it is disconnected from the transfer system 208 to prevent any residual fluid in the set 201 from leaking out. In some implementations, the proximal adapter 212 has a self-sealing access port, such as a pierceable septum, for covering the collection needle in the collection adapter 210, whether or not the collection needle is sealed with a boot or other cover. In other implementations, a separate side clamp, pinch clamp, or on/off valve on or connected with the tubing can be provided for the user to actuate prior to disconnecting the set 201 from the transfer system 208, as described in further detail below.

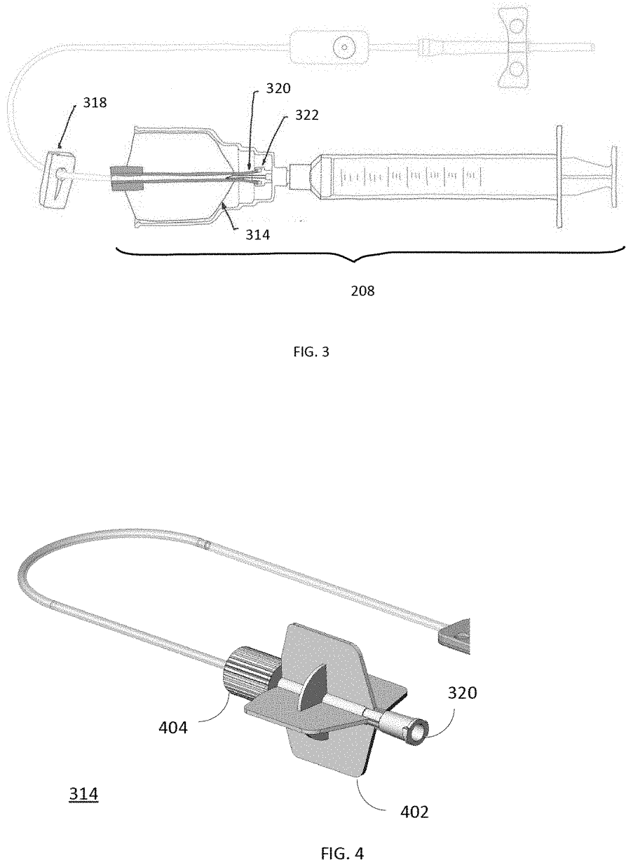

FIG. 3 illustrates the transfer system 208 with a proximal adapter 314. The proximal adapter 314 includes two or more support ribs that extend from a central fluid conduit, the support ribs being sized and configured to at least some of the inner dimensions of the collection adapter, for supporting the proximal adapter 314 therein. The proximal adapter 314 can include a first connector for sealably connecting with a corresponding second connector at the base of the needle in the collection adapter. For instance, the proximal adapter 314 can have a female Luer connector 320, while the collection adapter can have a male Luer connector 322. Before, or during disconnection of the set from the transfer system 208, a slide clamp 318 is provided and can be activated by clamping on the proximal tubing of the set, such that when the set is disconnected from the transfer system 208, leaking of residual fluid in the set is minimized or completely inhibited.

FIG. 4 is a perspective view of the proximal adapter 314, showing four support ribs 402 extending from a central fluid conduit, which terminates at a proximal end with a first connector 320 in the form of a female Luer 320. The proximal adapter 314 can have a grip 404 on a distal end opposite the first connector 320, for allowing a user to move, position and rotate the proximal adapter for connection with, and disconnection from, the collection adapter (not shown).

FIGS. 5(A) and 5(B) illustrates another implementation of a proximal adapter 502 as in FIGS. 3 and 4, but instead of using a clamp to seal the set upon disconnection with the transfer system, the proximal face of the proximal adapter includes a self-sealing septum 506, as shown in FIG. 5(B). The self-sealing septum 506 is pierceable by the needle in the collection adapter, whether or not the needle includes a pierceable boot 501 that covers the needle when the proximal adapter 502 is disconnected from the collection adapter, but which gets retracted away from the needle when the proximal adapter 502 is connected with the transfer system, as shown in FIG. 5(A).

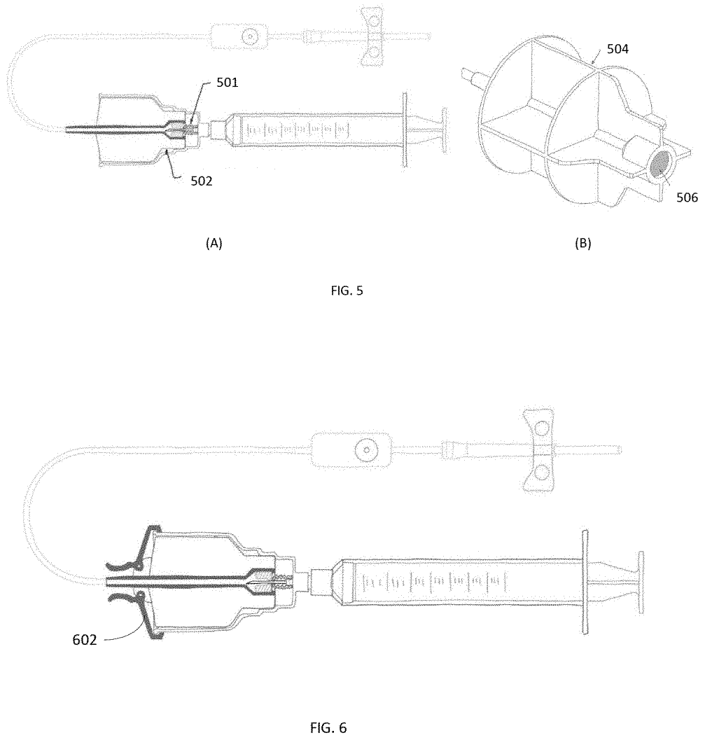

FIG. 6 shows another implementation of a proximal adapter having one or more clips 602 that clip around an outward-extending lip at the opening of the collection adapter, to secure the proximal adapter to the collection adapter and the proximal adapter's fluid conduit over the needle of the collection adapter. In this implementation, the proximal adapter can include a self-sealing septum, but does not require a connector such as a Luer connector, for example.

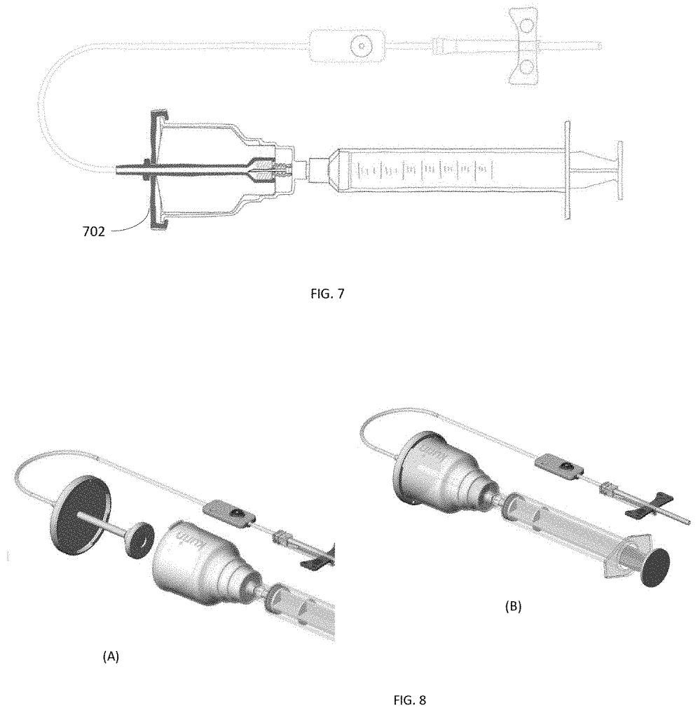

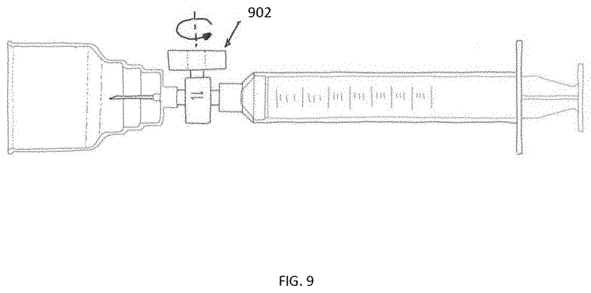

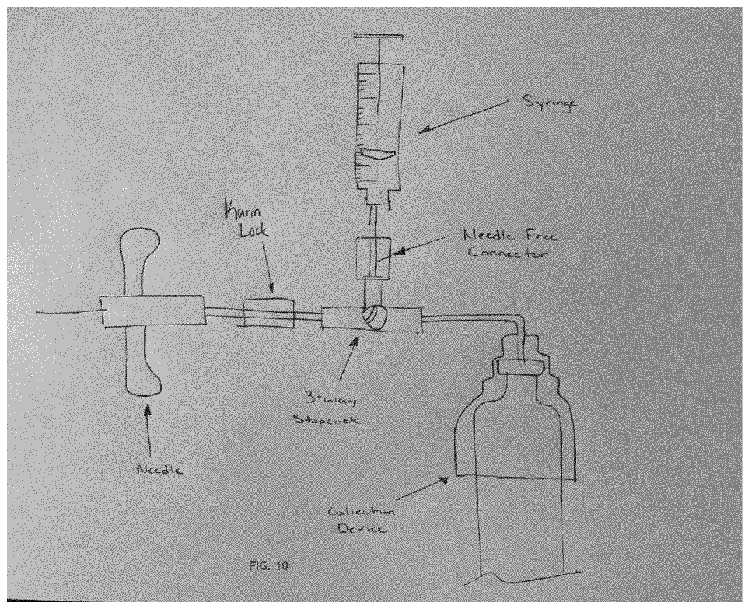

FIG. 7 shows another implementation of a proximal adapter, similar to FIG. 6, having a threaded ring 702 that is adapted to be twisted onto and off the collection adapter, for connection with and disconnection from the transfer system, respectively. FIGS. 8(A) and (B) show the proximal adapter of FIG. 7, both in its disconnected state (FIG. 8A) and in its connected state (FIG. 8B). The threaded ring eliminates the need for supporting ribs for inside the collection adapter.

In some implementations, turning the proximal adapter to connect and lock it to the collection adapter can actuate the opening of the fluid conduit through the proximal adapter, while turning the proximal adapter in a reverse direction to disconnect it from the collection adapter can actuate the closing or sealing of the fluid conduit. In these implementations, an external clamp or valve or other closure device is not needed.

However, instead of having a booted needle, the transfer system can have an external valve 902 to open and close flow between the syringe and the collection adapter, as shown in FIG. 9. The valve 902 can be a stopcock that a user rotates to open and close, a shuttle valve that can be shifted in one direction to open and shifted in the opposite direction to close, or a spring-loaded valve that is pushed to open, and then released to return to a closed position. Or any other type of fluid on/off valve.

In some implementations consistent with the subject matter described herein, a bodily fluid collection system, as described herein, can be used in the following manner. The user inserts the patient needle into the patient for blood collection. The insertion is part of a venipuncture that can occur in the patient's arm, or on another part of the patient's body. Once the fluid sample optimization device is primed, i.e. the first portion of the drawn sample is sidelined and at least temporarily contained in a contaminant containment chamber, the practitioner can draw subsequent portions of fluid samples by pulling back on the syringe plunger. Once the desired volume of fluid has been drawn and collected into the syringe, the patient needle is removed from the patient.

Next, the fluid collection set is removed from the transfer system that includes the syringe. The practitioner can then attach a collection bottle or tube to the collection adapter, to transfer fluid drawn into the syringe to one or more collection bottles or tubes, where a vacuum inside the bottle or tube pulls the appropriate volume into the collection bottle or tube.

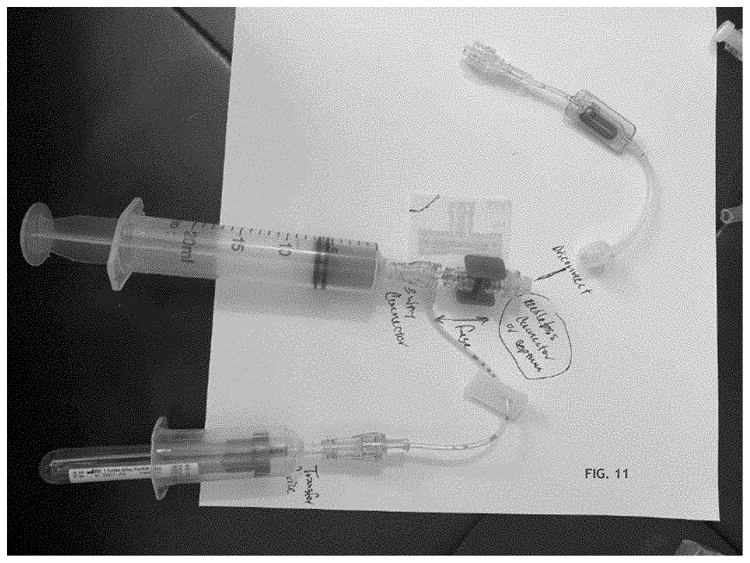

In some implementations, as shown in FIG. 10, a three-way stopcock with a needle-free connector can be added inline of, or between, a set of a needle and a fluid sample optimization device on a distal end of a fluid channel, and a collection device on a proximal end of the fluid channel. As shown in FIG. 10, there is no direct access from the needle to the collection device or vacuum provided thereby. In yet other implementations, as illustrated in FIG. 11, a three-way male connector (small picture) and a needleless connector can be added instead of the stopcock shown in FIG. 10. The needleless connector can include, without limitation, a Leur connection, a friction-fit connector, a threaded connector, or the like.

Although a few embodiments have been described in detail above, other modifications are possible. Other embodiments may be within the scope of the following claims.

1. A bodily fluid collection system for collecting a bodily fluid from a patient, the bodily fluid collection system comprising:
a patient needle for venipuncture of the patient to access a sample of the bodily fluid from the patient;
a fluid channel having a distal end connected with the patient needle to convey at least a sampling portion of the sample of the bodily fluid accessed from the patient;
a syringe having a barrel to contain bodily fluid, a plunger that can be activated to produce a vacuum in the barrel to draw at least some of the sampling portion of the bodily fluid into the barrel, and a hub opposite the plunger to create an inlet into the barrel;
a collection adapter having a connection port configured to securely connect with the hub of the syringe, and a collection interface opposite the connection port that creates a fluid conduit to the barrel via the connection port and the inlet of the hub; and
a proximal adapter connected to the proximal end of the fluid channel, the proximal adapter being configured to removably interact with the collection interface of the collection adapter to connect the proximal end of the fluid channel to the barrel via the fluid conduit and inlet to the barrel to allow conveyance into the barrel of the at least some of the sampling portion of the sample of bodily fluid.

2. The bodily fluid collection system in accordance with claim 1, wherein the fluid channel includes tubing.

3. The bodily fluid collection system in accordance with claim 1, further comprising a fluid sample optimization device provided on the fluid channel between the patient needle and the collection adapter, the fluid sample optimization device being configured to divert and sideline a first portion of the sample of the bodily fluid accessed by the patient needle, and to subsequently allow passage of the sampling portion of the sample of the bodily fluid accessed from the patient toward the collection adapter.

4. The bodily fluid collection system in accordance with claim 1, wherein the collection interface of the collection adapter includes a collection needle, the collection needle being self-sealing.

5. The bodily fluid collection system in accordance with claim 4, wherein the proximal adapter includes a self-sealing septum that is pierceable by the collection needle to secure the proximal adapter to the collection adapter.

6. The bodily fluid collection system in accordance with claim 1, wherein the collection interface of the collection adapter includes a first Leur interface, and wherein the proximal adapter includes a second Leur interface to removably interact with the first Leur interface.

7. The bodily fluid collection system in accordance with claim 4, wherein the collection adapter includes a housing to receive a vacuum-providing transfer bottle to connect to the collection needle and transfer at least a portion of the sampling portion of the sample of the bodily fluid from the barrel to the transfer bottle.

8. A bodily fluid transfer system for transferring a sampling portion of a bodily fluid collected by a syringe from a fluid channel, the syringe having a barrel to contain bodily fluid, a plunger that can be activated to produce a vacuum in the barrel to draw at least some of the sampling portion of the bodily fluid into the barrel, and a hub opposite the plunger to create an inlet into the barrel, the bodily fluid transfer system comprising:
a collection adapter having a connection port configured to securely connect with the hub of the syringe, and a collection interface opposite the connection port that creates a fluid conduit with the barrel via the connection port and the inlet of the hub; and
a proximal adapter connected to a proximal end of the fluid channel, the proximal adapter being configured to removably interact with the collection interface of the collection adapter to connect the proximal end of the fluid channel to the barrel via the fluid conduit and inlet to the barrel to allow conveyance into the barrel of the at least some of the sampling portion of the sample of bodily fluid, and upon removal of the proximal adapter from the collection interface, allow transfer of at least some of the sampling portion of the sample of the bodily fluid in the barrel to a second collector connected with the collection interface of the collection adapter.

9. The bodily fluid transfer system in accordance with claim 8, wherein the collection interface of the collection adapter includes a collection needle, the collection needle being self-sealing.

10. The bodily fluid transfer system in accordance with claim 8, wherein the collection interface of the collection adapter includes a first Leur interface, and wherein the proximal adapter includes a second Leur interface to removably interact with the first Leur interface.
